(19) United States
(12) Patent Application Publication
(10) Pub. No.: US 2014/0107510 A1
(43) Pub. Date: Apr. 17, 2014

(54) AUTOMATED ANALYSIS OF MULTI-LEAD ELECTROCARDIOGRAM DATA TO IDENTIFY THE EXIT SITES OF PHYSIOLOGICAL CONDITIONS

(71) Applicant: THE REGENTS OF THE UNIVERSITY OF MICHIGAN, Ann Arbor, MI (US)

(72) Inventors: Frank Bogun, Ann Arbor, MI (US); Clayton Scott, Ann Arbor, MI (US); Alfred Hero, Ann Arbor, MI (US); Tzu-Yu Liu, Ann Arbor, MI (US)

(73) Assignee: THE REGENTS OF THE UNIVERSITY OF MICHIGAN, Ann Arbor, MI (US)

(21) Appl. No.: 14/047,653

(22) Filed: Oct. 7, 2013

Related U.S. Application Data
(60) Provisional application No. 61/710,440, filed on Oct. 5, 2012.

(51) Int. Cl. A61B 5/0464; A61B 5/04

(52) U.S. Cl. CPC A61B 5/0464; A61B 5/04012
USPC 600/516; 600/523; 600/515; 600/518

(57) ABSTRACT

Techniques identify origins of ventricular arrhythmias (e.g., ventricular tachycardia or premature ventricular complexes) including exit sites or other sites using a single or multi-lead electrocardiogram (ECG) assembly. The ECG assembly is used to map an organ into a series of different three-dimensional (3D) regions. Pace maps or ventricular arrhythmia signals are used in form of ECG signals along with a supervised learning methods to pinpoint the potential origin of VT, i.e., exit sites, in the various regions.

### CROSS-REFERENCE TO RELATED APPLICATION

This application claims the benefit of U.S. Application Ser. No. 61/710,440, filed Oct. 5, 2012, entitled “Automated Analysis of Multi-Lead Electrocardiogram Data to Identify the Exit Sites of Physiological Conditions,” which is hereby incorporated by reference in its entirety.

### BACKGROUND

Myocardial infarction or acute myocardial infarction (AMI), commonly known as a heart attack, results from the interruption of blood supply to a part of the heart, causing heart cells to die. This is most commonly due to occlusion (blockage) of a coronary artery following the rupture or introduction of a vulnerable atherosclerotic plaque. The resulting restriction in blood supply and ensuing oxygen shortage, if left untreated for a sufficient period of time, can cause damage or death (infarction) of heart muscle tissue (myocardium). The presence of scar tissue can cause life threatening arrhythmias.

One particular arrhythmia associated with infarction is ventricular tachycardia (VT), which is a fast heart rhythm that starts in the ventricles (i.e., the lower heart chambers). While VT is a regular, albeit fast regular rhythm, if left untreated, some forms of VT may get worse and lead to ventricular fibrillation, which is a fast and irregular beating that can result in death. In ventricular fibrillation, the heart beats are so fast and irregular that the heart stops pumping blood, which can causes cardiovascular collapse and death. As to the causes of VT, they are numerous. In most cases, VT is caused by heart disease, such as a previous heart attack, a congenital heart defect, hypertrophic or dilated cardiomyopathy, or myocarditis or no detectable heart disease at all (a common condition called idiopathic VT).

There are different forms of VT including monomorphic VT (where all beats have the same shape), polymorphic VT (where the shape of different beats varies), pleomorphic VT (where one monomorphic VT changes into another monomorphic VT), ventricular flutter (a fast monomorphic ventricular tachycardia where beginning and end of individual beats cannot be clearly identified, e.g., sine wave shape) and ventricular fibrillation (a very rapid irregular VT with changing morphology of the individual beats). Premature ventricular complexes (PVCs) are also ventricular arrhythmias that occur as single beats during a baseline rhythm. PVCs can occur in patterns of every other beat or every third beat etc. They are very common even in the healthy subjects and may occur in up to 75% of the general population. While most often these PVCs are infrequent, they can occur frequently and may cause cardiomyopathy (weakening of the heart muscle). The ventricular arrhythmias can be eliminated or even cured by ablation treatment. There are numerous techniques for identifying the origin of these arrhythmias. Some of these techniques include multi-lead electrocardiagram measurements of electrical response to electrical stimuli applied to the heart, measuring various indicators such as heart beat rhythm or arrhythmia.

Once a location for VT or PVCs has been determined, then a physician may act accordingly, e.g., by applying an ablation treatment to the area that results in the patients arrhythmia. While there are techniques to assess the origin of VT, the techniques are limited in pinpointing, with sufficient accuracy, the particular tissue or regions of the heart that cause the VT.

### SUMMARY OF THE INVENTION

The present application describes techniques used to identify the site of origin of ventricular arrhythmias including VT exit sites and other sites resulting in ventricular arrhythmias. The techniques can use a multi-lead electrocardiogram (ECG, also referred to herein as EKG) assembly during or prior to performing a medical procedure on a patient. The ECG assembly may be used to map the heart (or other bodily organs) into a series of different three-dimensional (3D) regions or localization units or metrics (i.e., coordinates), of differing sizes and contours to the heart, determined by algorithm analysis of multi-lead ECG data. The localization metrics are defined by pace maps, which are signals generated by catheter stimulation in defined locations within the heart to specifically pinpoint the potential sources of ventricular arrhythmias, (PVCs, VT, polymorphic VT, pleomorphic VT, ventricular flutter or ventricular fibrillation) i.e., VT origins or exit sites or other useful sites. Non-stimulated signals can also be used as reference signals analog to pace-mapping signals. The size of these localization units may be determined by an algorithm analysis, to increase accuracy of VT exit site measurement and to increase sensitivity and specificity for receiver operator characteristics curves (ROC). The size of the localization units may be reported as coordinates, areas or volumes. The present application describes algorithm-based optimizations that greatly enhanced the accuracy of ECG determination of the origin of ventricular arrhythmias.

In accordance with an example, a method for identifying a physical characteristic and localizing characteristics of sites within a bodily organ, the method includes: collecting electrocardiogram derived signals from a plurality of multiple electrical leads, each lead positioned to collect a respective electrocardiogram signal from different sites of the bodily organ; comparing the collected electrocardiogram derived signals to reference signal data to identify which anatomical region among a predetermined set of anatomical regions corresponding to the bodily organ contains the physical characteristic, wherein the reference signal data is determined from previously-labeled electrocardiogram derived signals for one or more different anatomic areas of the bodily organ; and using mapping data derived from the subsequently collected electrocardiogram derived signals to determine a sub-region within the anatomical region that contains the physical characteristic.

In accordance with another example, an apparatus includes: a computer processor; and a memory storing computer-readable instructions that, when executed by the computer processor, cause the computer processor to, collect electrocardiogram derived signals from a plurality of multiple electrical leads, each lead positioned to collect a respective electrocardiogram signal from different stimulated sites of the bodily organ, compare the electrocardiogram derived signals to reference signal data to identify, among a set of predetermined anatomical regions of the bodily organ, anatomical regions that contain a physical characteristic of the bodily organ, wherein the reference signal data is determined from previously-labeled electrocardiogram derived signals for one or more different anatomic areas of the bodily organ, and use mapping data derived from the electrocardiogram derived signals to determine a sub-region within the anatomical region that contains the physical characteristic.

### BRIEF DESCRIPTION OF THE DRAWINGS

This patent or application file contains at least one drawing executed in color. Copies of this patent or patent application publication with color drawing(s) will be provided by the United States Patent and Trademark Office upon request and payment of the necessary fee.

FIG. 1 illustrates cardiac sections of A-J serving as the regions to which the VT exit sites were assigned

FIG. 2 depicts a confusion matrix comparing accuracy data in the 34 patients in whom the testing data were obtained.

FIG. 3 is a plot of the median of electrocardiogram signals from 12 different leads for the regions A-J of FIG. 1, based on all the pace-maps collected in different regions.

FIG. 4A is an electroanatomical map of the inferior aspect of the left ventricle in a patient with a large inferior wall infarction. The low voltage area was 242 cm². Area J (tags measuring 17.4 cm²) and I (tags measuring 6.7 cm²) are displayed. Location of the left ventricular apex and mitral valve annulus (MVA) is indicated. FIG. 4B is an electroanatomical map of sites where pace-mapping was performed (additional tags).

FIG. 5 is an illustration of a system for performing VT exit site assessment and assignment in accordance with an example, including showing a pace-mapping module.

FIG. 6 is a flow diagram of an example technique for VT exit site assessment and assignment.

### DETAILED DESCRIPTION

The present application describes techniques for constructing and displaying mapped points within an imaged bodily organ using electrocardiogram leads for stimulation and tracking. In an example, the techniques have been used to construct and map ventricular tachycardia (VT) sites in the heart, using a single or multi-lead electrocardiogram device and pacing. The bodily organ, e.g., the heart, may be mapped into different anatomical regions (also termed areas or segments interchangeably herein) and each anatomical region may be sub-divided into different sub-regions or sub-areas and coordinates to identify VT exit (or origin) sites, using a signal analysis method, e.g., supervised learning method of analysis of digitized pace-map morphologies combined with pacing sites as training data. The term “region” or “area” within this description is used to identify a location of interest within the heart or a bodily organ that may be represented as x/y/z/coordinates or a site, an area or a volume or any 3-dimensional structure. Furthermore, reference is made herein to VT exit sites in describing numerous examples. That reference is intended to include exit sites, origin sites and other sites for any type of ventricular arrhythmias, such as PVCs, VT, polymorphic VT, pleomorphic VT, ventricular flutter or ventricular fibrillation.

Electrocardiogram signals from the different leads may be measured and analyzed from different regions; and differentiating characteristics are established allowing one to differentiate signal defining characteristics from one anatomical region, sub-region, or coordinate from another. The signals may originate from single or multi-lead ECGs or electrogram tracings from implanted devices (implanted cardioverter defibrillator or pacemaker or other implanted devices) or non-implanted devices.

The number of sub-regions may be determined based on preferences of the operator and may include characteristics like coordinates or different anatomic localization characteristics (i.e., right vs left ventricular, epicardial vs endocardial vs intramural, different endocardial regions, different epicardial regions, pulmonary artery, aortic cusps, papillary muscles, etc.). In some examples, the positions and dimensions of the regions and sub-regions may be defined relative to one another, e.g., using relative coordinates between regions and sub-regions. In some examples, the positions and dimensions may be determined based on fixed coordinates, e.g., a universal coordinate system external to the bodily organ. In some examples, the positions and dimensions are determined from transformations, e.g., to account for differences in patient physiology, differences in sizes of bodily organs across patients, differences in the orientations of the bodily organ upon data collection, and/or differences in the state of the bodily organ.

The accuracy of placement of a measured electrocardiogram signal into localization of a VT exit site or site of origin or critical component may be enhanced by comparing and adding one or more adjacent regions for increased receiver operating characteristic (ROC) performance. With the anatomical regions and sub-regions and coordinates identified, the present techniques may be combined with different imaging techniques; and they may be used in numerous applications, including cryogenic and radiofrequency or other catheter treatment techniques, with various controllable degrees of accuracy. The accuracy may be adjusted, for example, by adjusting the number of regions, the number of sub-regions within each region, and/or the number and placement of lead electrodes in or around the bodily organ. Furthermore, the size of the anatomical regions may be adjusted depending on the likely number of VT exit sites, the determined number of VT exit sites, the proximal grouping of those VT exit sites, or other metrics. By using a library of signals and anatomic locations generated by predetermined regions of interest the present application is able to provide greater flexibility in algorithm learning and more accurate VT exit site identification.

In an example implementation, 34 post-infarction patients were examined and pace-mapping was performed from within scar tissue. A computerized algorithm that used a supervised learning method (e.g., implemented through support vector machines, relevance vector machines, neural networks, and/or logistic regression classifiers) received the digitized pace-map morphologies combined with the pacing sites as training data. For confirmation purposes, no other information (i.e., infarct localization, bundle branch block morphology, axis or R wave pattern) was used in the algorithm. The training data were validated in 58 VTs in 33 patients, where only the pace-map and/or VT morphologies were used. The sizes of 10 different predefined anatomical regions within the heart were used to generate the electrical signals (so called pace-maps) as the determining factor. Accuracy was found to be 69% for pace-maps and when 2 adjacent regions were combined, accuracy improved to 88%. Validation of the data in 33 patients showed an accuracy of 71% for localizing a VT exit site to one of the 10 regions within the left ventricle. If combined with the best adjacent region, accuracy improved to 88%. The median anatomic size of each section was 21 cm² in an example. The median spatial resolution of the 12-lead ECG pattern of the pace-maps for a particular region was 15 cm².

### Example

### Patient Characteristics

### Table 1

Table 1 provides characteristics of the 34 patients examined in an example study for training data purposes. Mapping and radiofrequency ablation were performed in a consecutive series of 34 patients (31 males, age: 68±9 years, ejection fraction: 0.25±0.12) with post-infarction VT. Left ventricular pace-mapping was performed in these patients. Eighteen patients (53%) had a prior inferior, 9 (26%) had an anterior and 7 (21%) had an inferior and anterior wall myocardial infarction. Pace-maps were collected in these patients and used as training data. The training data were subsequently (see below) validated in another 33 consecutive post-infarction patients (32 males, age 67±10 years, ejection fraction: 0.26±0.12).

TABLE 1
Characteristics of Patients used for Training and Validation of Data

| | | Validation | |
|---|---|---|---|
| Variable | Training data | data | p-value |
| Patients, n | 34 | 33 | |
| Induced VTs, n | 8 ± 4 | 8 ± 5 | 0.71 |
| Age, years | 68 ± 9 | 67 ± 10 | 0.63 |
| Male/female, n | 31/3 | 32/1 | 0.61 |
| Left ventricular ejection fraction, % | 25 ± 12 | 26 ± 12 | 0.86 |
| Location of myocardial infarction | | | 0.18 |
| Anterior | 9 | 3 | |
| Inferior | 18 | 21 | |
| Anterior and inferior | 7 | 9 | |
| Scar size, cm² | 80 ± 31 | 86 ± 35 | 0.42 |
| VT cycle length, msec | 384 ± 115 | 370 ± 89 | 0.28 |
| Patients on amiodarone, n | 24 | 18 | 0.21 |

Abbreviations:
VT = ventricular tachycardia.

### Mapping

For the procedure, initially, a 6 Fr quadripolar electrode catheter was introduced into a femoral vein and positioned at the right ventricular apex. Next, programmed right ventricular stimulation was performed with 1-4 extrastimuli to induce VT. For convention purposes, sustained VT was defined as VT lasting >30 seconds or requiring termination secondary to hemodynamic compromise of the patient. Next, Electroanatomical mapping (CARTO™, Biosense Webster, Inc., Diamond Bar, Calif., USA) was performed with a 3.5-mm-tip, open-irrigation ablation catheter (Thermocool Navistar, Biosense Webster). The resulting electrogram signals, i.e., ECG signals, were applied to a signal processing pre-stage, namely bandpass filtering at 50-500 Hz. The intracardiac electrograms and leads V1, I, II and III were displayed on an oscilloscope and recorded at a speed of 100 mm/sec; and the recorded data was stored on an optical disc (EPMed Systems, West Berlin, N.J., USA).

From the ECG signals, a voltage map of the left ventricle was generated during sinus rhythm. Low voltage was defined as a bipolar voltage amplitude ≦1.5 mV (e.g., Marchlinski F E, Callans D J, Gottlieb C D, Zado E. Linear ablation lesions for control of unmappable ventricular tachycardia in patients with ischemic and nonischemic cardiomyopathy, Circulation, 2000; 101:1288-1296). For left ventricular mapping, a bolus of 5000 units of heparin was administered and additional heparin was administered to obtain an activated clotting time >300 seconds.

### Mapping

With the voltage map of the left ventricle formed, pace-mapping was performed from within the low-voltage area. The cycle length of pace-mapping was that of the targeted VTs. Pace-mapping was performed uniformly throughout the low voltage area at distinct sites where the local electrogram signals differed from those of the prior mapping site. This allows for pacing from different coordinate sites to build up a signal library for validation purposes. At least 2 consecutive capture beats were required to include the pace-map in the analysis. Low voltage or scar was defined as <1.5 mV (see, e.g., Marchlinski F E, Callans D J, Gottlieb C D, Zado E. Linear ablation lesions for control of unmappable ventricular tachycardia in patients with ischemic and nonischemic cardiomyopathy, Circulation, 2000; 101:1288-1296). Pacing was performed at an amplitude of 10 mA at a pulse width of 2 msec. The pacing cycle length was the mean cycle length of the induced VTs in a given patient.

### Data Analysis—Determination of Training Data

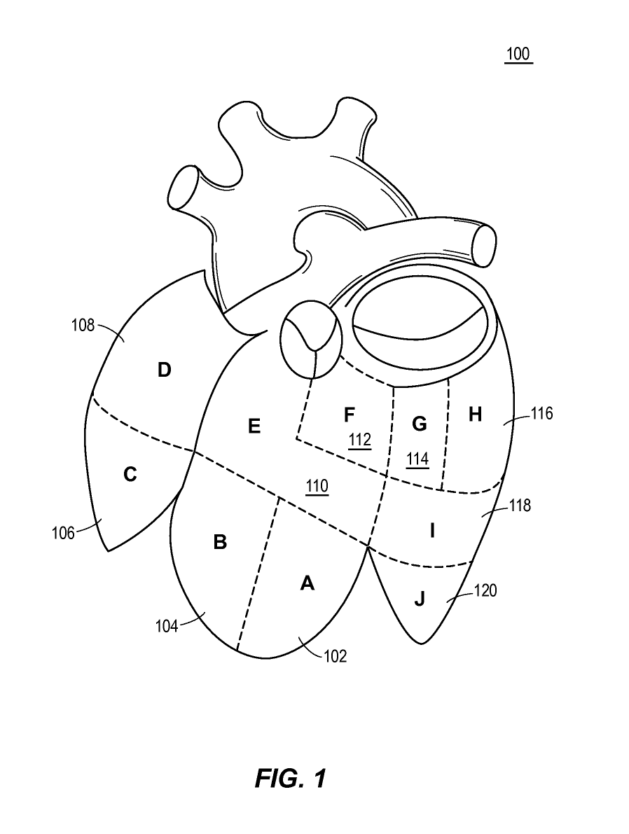

FIG. 1 shows a bodily organ 100 that has been segmented into 10 regions, 102-120, that were formed for a heart from digitized 12-lead ECGs generated within low voltage tissue, as analyzed in a customized MatLab program (MathWorks, Inc., Natick, Mass., USA). The regions were assigned a particular anatomic location within the heart based on a previously-described schema, A-J (102-120, respectively), FIG. 1. The assignments were made in this example in accord with that of conventional techniques, (see, e.g., Miller J M, Marchlinski F E, Buxton A E, Josephson M E. Relationship between the 12-lead electrocardiogram during ventricular tachycardia and endocardial site of origin in patients with coronary artery disease, Circulation, 1988; 77:759-766,).

To determine the regions a schema was used following these guidelines: The distance between an apex and a base was divided into 3 equal segments (basal, mid and distal). Regions A (102), B (104), C (106), and J (120) were the distal segments; regions I (118), E (110), and D(108) were the mid segments; and regions E (110), F(112), G(114), and H (116) were the basal segments. Localization was performed by 2 independent observers. Discrepancies were resolved by consensus. There was an 85% inter-observer agreement and a kappa value of 0.84, indicating that the inter-observer variability was low, i.e., the inter-observer agreement was high.

The accuracy of the pace-mapping and identification of the particular region corresponding to a VT exit site is determined based on the size of the region that generates a similar electrocardiographic signal. The signals used to generate the electrogram testing signals were ˜750, however there is no limit and inclusion of more signals will increase accuracy. It was determined that the spatial resolution for post-infarction VT exit sites using ˜750 testing signals was ˜10-15 cm². The number of sub-regions may vary across the anatomical regions A-J (102-120).

The regions A-J (102-120) may change in scale or number (with smaller or larger numbers of regions) for different procedures, and for different patients. Furthermore, the regions may be clustered in pairs, triples, quartets, etc. as discussed herein. In other examples, the regions may be clustered in other ways, with other numbers of adjacent regions, to assist in VT exit site mapping. The regions may be described as anatomic areas, anatomic volumes or anatomic coordinates. They can be displayed in different hierarchical manners depending on the operator's preferences (i.e., VT origin, endocardial, region D, (108), etc.). A correlation with imaging methods using echocardiography, magnetic resonance imaging with and without contrast, cardiac tomography may be necessary to correlate and adapt signals from and between different patients.

It is noted that the present examples are described in reference to a 12-lead ECG setup. However, any number of leads may be used. Indeed, to achieve considerably higher spatial resolution, increasing the number of leads to 64, 128, 256 or more could be used. Furthermore signals from implanted devises (implanted cardioverter defibrillator or pace maker or other implanted devices) or non-implanted devices can also be used for analysis.

We wanted to assess whether a computerized algorithm is able to distinguish the pacing site based on QRS morphology of the 12-lead ECG. To achieve this, a supervised learning method (e.g., customized support vector machine (SVM)) was used. It is noted that in other examples, any part of the ECG signal, P-wave, QRS complex, T wave, or any combination of intervals (i.e., PQ, QT, ST, etc.) or intervals themselves or signals obtained during arrhythmias may be used instead of a QRS signal.

SVM is a machine learning technique. In binary classification problems (i.e., separation of digitized ECG signals originating from 2 regions), the principle of SVM is to find a hyperplane (i.e., a separation of signals in a multidimensional space) to separate the signals from the 2 regions, such that the separation between the two classes is maximized. This is only one example of a signal analysis technique and other analysis techniques may be used to compare the electrogram signals. While a machine learning technique is described, any signal analysis technique may be used, including un-supervised learning models.

In this multiclass problem (e.g., assignment of ECG signals into 10 different regions), we trained a classifier using a one-against-one approach to break the multiclass classification into several binary problems. In the case of 3 regions, for example, the QRS complex of the 12 lead ECG may be classified as a signal associated with region A (102), B (104), or C (106) by aggregating the outcomes of the 3 binary tests: region A (102) vs B (104), B (104) vs C (106) and A (102) vs C (106). In the case of 10 ECG regions there will be C₂¹⁰=45 comparative tests performed for each set of ECG signals. We trained the algorithm for each pair of two different regions where the 12 lead ECG signals originate from and analyzed how often a particular region was chosen by the algorithm to determine which region (A-J, 102-120) an ECG signal would be assigned to.

The algorithm was trained by providing it with the pace-map location data and ECG data. The training data included the digitized 12-lead QRS morphology of the pace-maps and the locations of the regions (region A-J, 102-120).

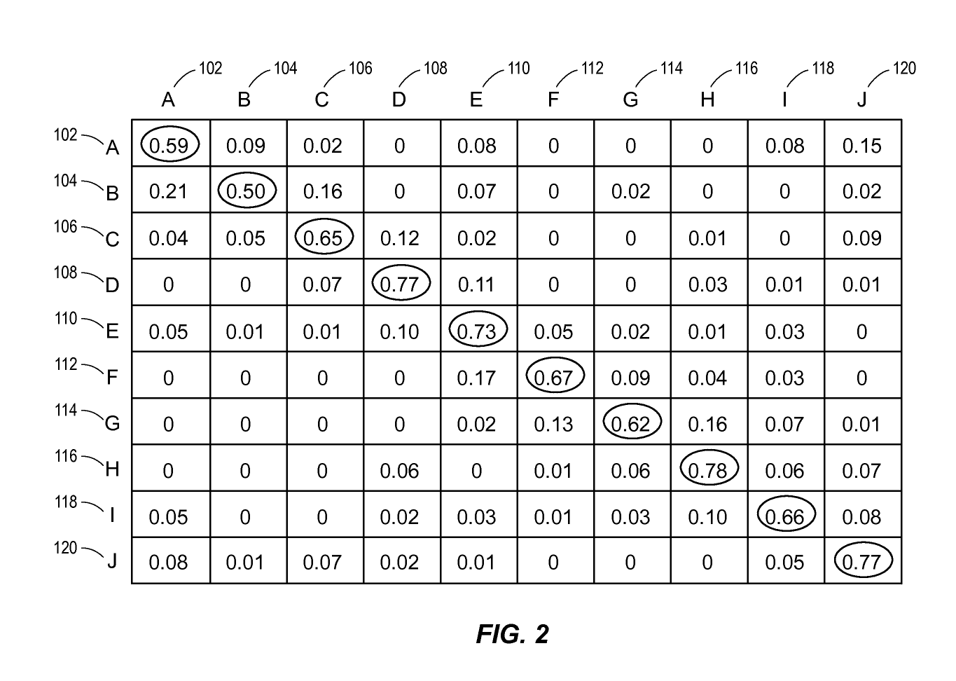

The algorithm to assign a particular pace-map to a particular region (A-J, 102-120) was cross-validated with the pace-map data. The results were displayed in a table form (confusion matrix) indicating the percentage of correctly classified data (the ringed data indicate percentages of the correctly identified data in FIG. 2). The training data containing pace-maps only were then validated using 58 VTs from 33 different patients where exit sites were identified by pace-mapping.

The algorithm was designed to determine which stimulus-QRS interval of the pace-maps was optimum for pace mapping. The stimulus-QRS internal varied from 12 ms to 390 ms. It is possible that very long stimulus-QRS intervals do not reflect a site where local capture of the myocardium occurred but instead indicate activation of protected areas that can result in generation of a QRS complex remote from the pacing site. We wanted to determine the stimulus-QRS complex interval that resulted in the highest accuracy of pace-maps. To achieve this, the accuracy of the SVM analysis was analyzed by 10 msec intervals of the stimulus-QRS interval starting from 30 msec. A total of 1163 pace-maps were analyzed. The highest accuracy (69%) was identified with a stimulus-QRS interval ≦70 msec. For example, a stimulus-QRS interval ≦70 msec was present in 774 pace-maps.

Because of a high probability of overlap of a particular ECG morphology originating from one region with a neighboring region, we used an algorithm ranking the best correlating regions to assess how often a particular region needs to be visited to be in the correct region. Accuracy was arbitrarily defined as a pace-map morphology that was correctly assigned to either the best or second-best matching sector.

### Data Analysis—Determination of Spatial Resolution of the 12 Lead ECG Pattern Based on Anatomic Region (Table 2)

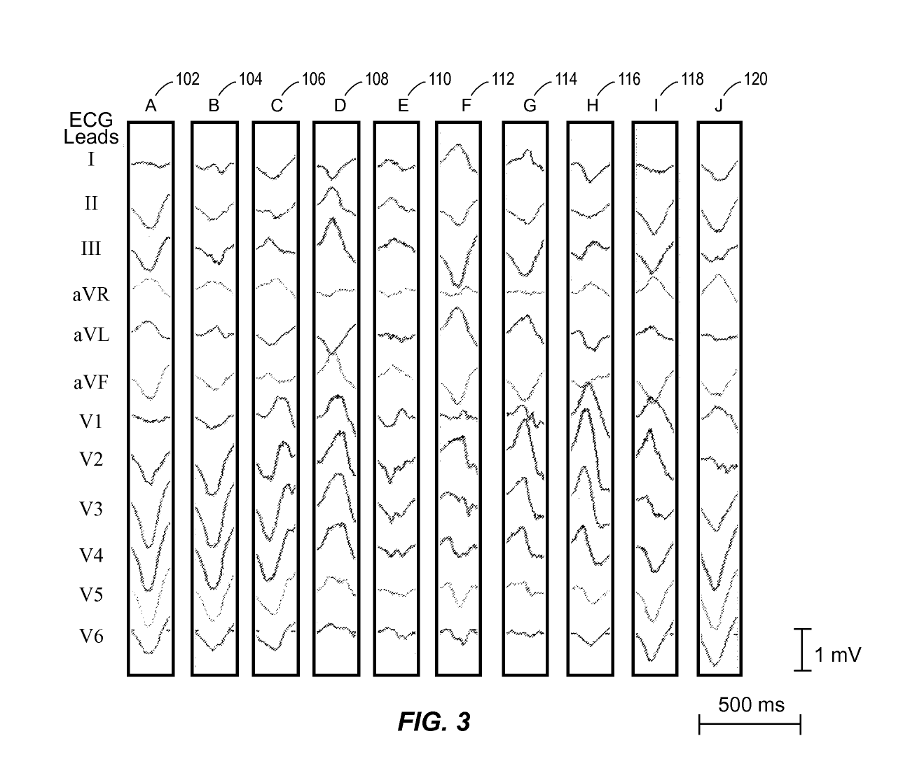

The size of the anatomical regions (A-J, 102-120) that generated a particular ECG morphology during pace-mapping within low-voltage tissue was determined. Table 2 depicts anatomic area and spatial resolution data, taken as medium values, for areas A-J, 102-120, in an example. A median of the 12-lead ECG signals was generated based on the pace-maps of a particular region, as taken from the ECG signal data of FIG. 3. The median 12-lead ECG morphology was then used as a template signal that was compared to the pace-maps assigned to this and other regions and a correlation coefficient was generated. By taking the median, the data plots in FIG. 3 represent a smoothed data set collected from each of the 12 leads. The signals measured by each lead corresponding to each region are provided.

TABLE 2
Anatomic Areas and Spatial Resolutions of the ECG

| | Region | | | | | | | | | |
|---|---|---|---|---|---|---|---|---|---|---|
| | A | B | C | D | E | F | G | H | I | J |
| Anatomic | 13 | 13 | 22 | 62 | 26 | 19 | 21 | 22 | 30 | 21 |
| Area, cm² | (12-15) | (10-17) | (20-29) | (57-69) | (23-32) | (16-23) | (17-23) | (19-30) | (27-32) | (19-25) |
| Spatial | 15 | 20 | 18 | 20 | 16 | 14 | 10 | 11 | 10 | 18 |
| resolution, | (11-20) | (15-21) | (9-25) | (14-27) | (9-26) | (7-23) | (5-21) | (6-17) | (8-21) | (12-28) |
| cm² | | | | | | | | | | |
| Accuracy | 0.59 | 0.5 | 0.65 | 0.77 | 0.73 | 0.67 | 0.62 | 0.78 | 0.66 | 0.77 |

### Anatomic Area and Spatial Resolution Data are Displayed as Medium Values and (Interquartile Range)

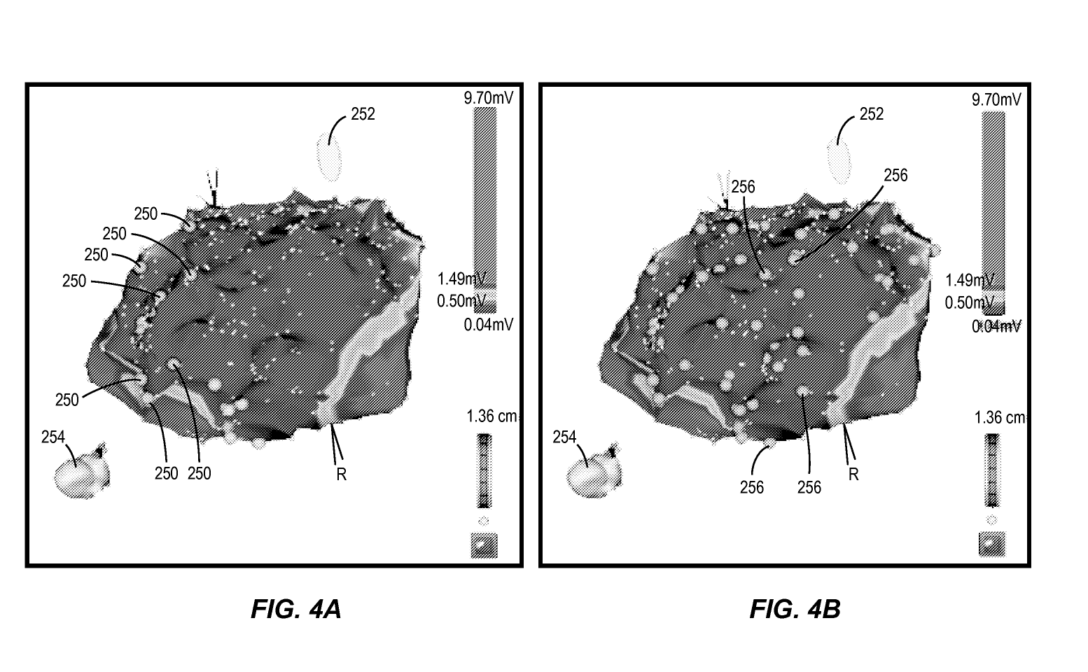

The spatial resolution of such a region was determined based on receiver operator characteristics (ROC) curves that generated a cut-off value separating the median ECG electrogram of a region (A-J, 102-120) from pace-maps of other regions. This was done for each patient in the low voltage area where pacing was performed. The gold-standard was whether or not a pace-map belonged to a particular region or not. Once the cut-off value was determined for each region, the area encompassing sites with a correlation coefficient equal to or greater than the cut-off value was measured on the electroanatomic map (FIG. 4A or 4B). The spatial resolution then was averaged for all patients (i.e., the spatial resolution of area A from a given patient was averaged with the spatial resolution of area A from other patients) and the areas were reported per region.

### Data Analysis—Validation of Training Data

The 12-lead ECGs of 58 VTs from 33 post-infarction patients in whom the exit sites were determined by pace-mapping were analyzed prospectively. An exit site was defined as a site where the pace-map matched the targeted VT and where the stimulus-QRS interval was ≦30% of the VT cycle length when pacing was performed during sinus rhythm (see, e.g., Bogun F, Bahu M, Knight B, Weiss R, Goyal R, Daoud E, Man K, Strickberger S, Morady F. Response to pacing at sites of isolated diastolic potentials during ventricular tachycardia in patients with previous myocardial infarction, Journal of the American College of Cardiology, 1997, 30:505-513). The VTs of these 33 patients served as test data and the pace-maps from the initial 34 patients served as the training data. Data were analyzed for validation of accuracy of the computerized algorithm.

### Comparison to Other Algorithms

The computerized algorithm described was compared to a previously described algorithm using the pace-maps of the present study. Specifically, Miller, et al. (cited above) developed an algorithm based on infarct location, bundle branch type configuration, QRS axis and precordial R wave progression. Paced QRS morphologies obtained during pace-mapping within scar were analyzed for the site of origin using the algorithm described by Miller, et al. Segal et al. (Segal O R, Chow A W, Wong T, Trevisi N, Lowe M D, Davies D W, Della Bella P, Packer D L, Peters N S. A novel algorithm for determining endocardial vt exit site from 12-lead surface ecg characteristics in human, infarct-related ventricular tachycardia, J Cardiovasc Electrophysiol, 2007, 18:161-168) also described an algorithm to determine the location of a VT exit based on the 12 lead ECG morphology. We also compared the above specified method with the algorithm described by Segal et al.

### Statistical Analysis

Categorical variables were compared with the Chi square test or Fisher exact test, as appropriate. Continuous variables were compared with t-test. Continuous variables were reported as mean and standard deviation under the assumption that they were normally distributed.

A P-value <0.05 was considered significant. Receiver operator characteristics curves were generated to determine cut-off values for the spatial resolution of ECG patterns of particular anatomic regions. Median values of ECG signals from a particular anatomic region were displayed.

Accuracy of Correlation of an ECG Pattern with an Anatomic Region Based on Computerized Algorithm

A total of 774 pace-maps were used for this analysis. Overall accuracy of the SVM analysis for assigning a 12-lead ECG pace-map to an anatomical region was 69%. The accuracy varied from region to region (range: 50% to 78%). When accuracy was determined for the 2 top-ranked regions, it improved to 88%. In other words, the ability to determine the site of origin of an ECG pattern within one region or its neighbor was 88%. The overall rank to correctly identify a particular region was an average of 1.5 (i.e. an average of 1.6 regions needed to be assessed to assign a pace-map to the appropriate anatomical location).

Accuracy of Correlation of an ECG Pattern with an Anatomic Region Based on Published Algorithms

Overall accuracy using a previously described algorithm was 19% with a range of 7 to 54%. When the 12-lead ECGs of the VTs were used for validation, the algorithm described by Miller et al. (cited above) could be used in 41% of the VTs with an overall accuracy of 13% (range 0-100%). In the Miller et al. study, the algorithm could be applied to about 50% of post-infarction VTs. Similarly in the present study, Miller et al.'s algorithm could be applied to 46% of all the pace-maps. Segal et al.'s algorithm could be applied to 91% of the 12 lead VT ECGs but its accuracy was only 36%.

### Spatial Resolution of an ECG Pattern Depending on Anatomic Region

The median spatial resolution of pace-mapping for a post-infarction VT exit site was 15 cm² with a range of 10 cm² (area G) to 20 cm² (area D). The entire left ventricular endocardial area was 226±58 cm². The anatomic area of the sections ranged from 13 to 62 cm² with a median of 21 cm². The mean scar area in the patients in whom the mapping data were obtained was 85 cm².

### Validation of Training Data

Accuracy was determined using the 774 pace-mapping signals as the training data and 58 VTs as the testing data. The accuracy was 71% for assigning the testing data into the correct region (A-J). Overall accuracy increased to 88% for identification of a matching region if the 2 top-ranked regions were included. The overall rank to correctly identify a particular region was an average of 1.7.

Compared to a previously published algorithms based on visual inspection, the localizing value of the multi-lead (e.g., 12-lead) ECG improved substantially when a computerized algorithm was used. Determination of a VT exit site was automated without the use of a complicated algorithm. An accuracy of almost 90% was achieved when neighboring regions with overlapping ECG features were included. The spatial resolution of a VT-ECG pattern originating from post-infarction scar was a medium of 15 cm².

### Localizing Value of the 12-Lead ECG for Determining a VT Exit Site

Whereas conventional techniques attempting to localize analysis using a conventional 12-lead ECG setup have been lacking, with the present techniques, we demonstrated that an accuracy of up to 70% can be achieved when supervised learning methods are used. By automated analysis, objective data regarding the exit of VT can be generated before an ablation procedure. In contrast to previously described methods, our analysis does not require information such as infarct location, bundle branch block morphology, axis, or precordial R wave pattern. The spatial resolution of a particular VT-ECG has not been described in post-infarction patients. In this study, the area of each of 10 LV regions was measured and ranged from 13-62 cm² with a medium of 21 cm². Interestingly, the spatial resolution of the region accounting for a particular ECG pattern was smaller and ranged from 10-20 cm² with a medium area of 15 cm². This suggests that the ECG of VT contains more localizing information than previously thought. The spatial resolution of pace-mapping within scar was reported to be in the 0-17 cm² range¹⁰ and therefore also is within the range of the spatial resolution of the 12-lead ECG.

Accuracy Variation with Region

A previously-used algorithm had an accuracy >70% for predominantly apical septum regions (A 102, B 104). These were the regions that performed worst on the computerized analysis, suggesting that these algorithms are complementary and that the automated algorithm can be further improved. With the previous algorithm, more than 50% of the VTs did not fit any particular pattern and therefore could not be classified, making the algorithm impractical. The main limitation of that previous algorithm was the lack of applicability for patients with prior anterior wall infarctions and for right bundle branch block VT morphologies. In contrast, the computerized algorithm performed best in regions affected by anterior wall infarcts (e.g., Region D 108). The discriminatory value of the computerized algorithm was imperfect in the apical septum area where the accuracy was around 50%. However, if 2 zones are combined, the accuracy for determining the larger sector improved to approximately 70% in these regions.

Because there are no clear-cut demarcations between left ventricular regions and because infarct scars are not necessarily confined to one region, it seems appropriate to use a ranking classification indicating the best and second-best matching regions for test data. In order to identify a VT exit region (A-J, 102-120) based on the 12-lead ECG, an average of 1.6 scan attempts was needed to get to the correct region. Because the mean size of the ECG-determined region is approximately 15 cm², combining 2 regions results in an area of 30 cm² in which more than 80% of VT exit sites could be assigned to. The mean scar area in the patients in whom the mapping data were obtained is a mean of 85 cm², the 12 lead ECG helps to narrow down the area of interest to approximately ⅓ for >80% of VTs.

### Clinical Implications

As shown with the discussed example, automated identification of a VT exit site based on the 12-lead ECG of post-infarction VT was possible with an accuracy of about 70% for identifying a region of interest with a size of approximately 15 cm², although the present techniques are not limited to a particular accuracy range. In any event, as described, identification of an area of interest up-front will help to facilitate mapping and ablation of complex post-infarction VTs, especially in patients with large scars.

In another analysis of 45 patients, we assessed accuracy of a computer algorithm that used a Gauss mixture model, which is an unsupervised learning method to identify whether a ventricular arrhythmia was originating from the endocardium or the epicardium. In order to achieve this, training data from 3459 sites (1828 epicardial and 1631 endocardial sites) were obtained and features of the QRS signals were identified by the computer algorithm that helped to distinguish epicardial from endocarial origins with an accuracy of 79%. In healthy individuals the accuracy was >90%. This compares to an accuracy of 69% when conventional algorithms are used. Distinction of epicardial vs endocardial origins of ventricular arrhythmias is of key importance since an epicardial mapping and ablation procedure is much more invasive than an endocardial procedure.

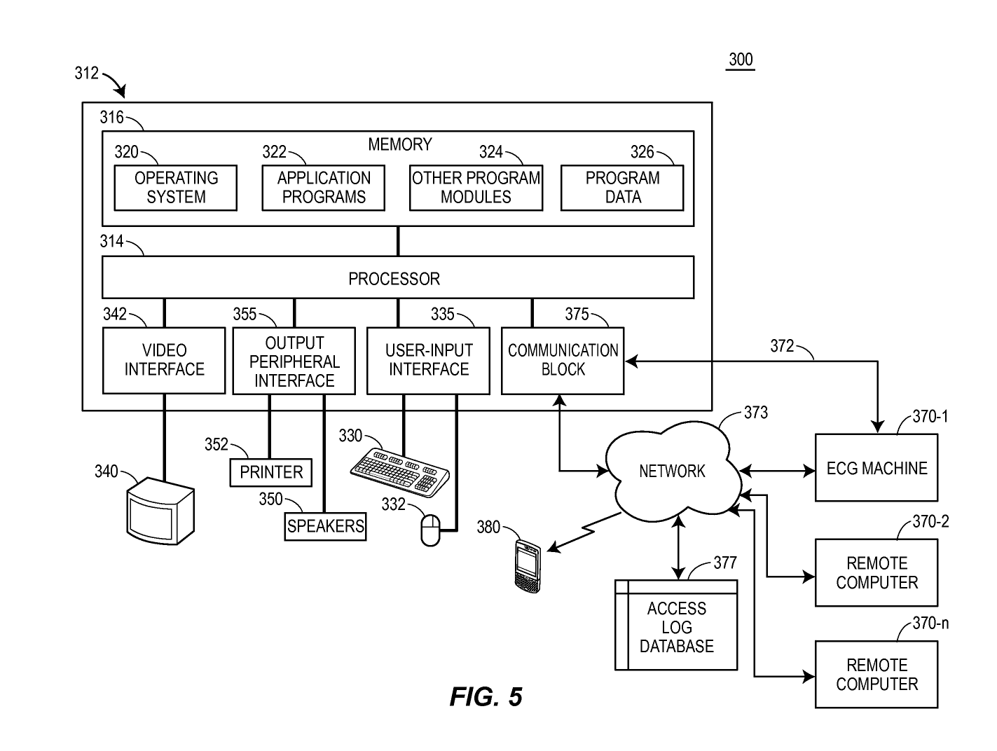

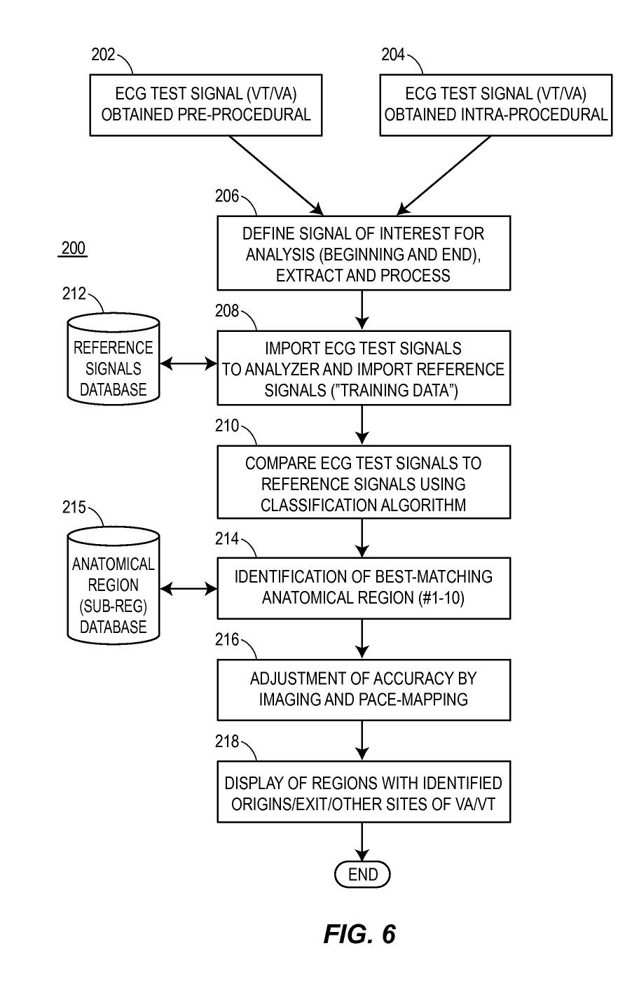

FIG. 5 illustrates an example computer system for performing ventricular arrhythmia/VT analysis and pace-mapping in accordance with the examples hereinabove. The techniques described above (and in FIG. 6) may be coded, in software, hardware, firmware, or combination thereof, for execution on a computing device such as that illustrated in FIG. 5. Generally, FIG. 5 illustrates an example of a suitable computing system environment 300 to interface with a medical professional or other user to analyze medical data, in particular electrocardiogram (ECG) signals captured at the point of assessment or from a stored database of historical ECG signals. It should be noted that the computing system environment 300 is only one example of a suitable computing environment and is not intended to suggest any limitation as to the scope of use or functionality of the method and apparatus of the claims.

FIG. 6 illustrates an example process 200 for analyzing ventricular arrhythmia/VT exit sites in accordance with the foregoing. Initially, data collection and definition of parameters are performed; the data may be recorded pre-procedurally (202) or intra-procedurally (204) (referring to the electrophysiology procedure with or without mapping and ablation). Pre-procedural signal acquisition may come from single or 12 lead (or other multi-lead) ECG tracings; while in other examples, electrograms may be obtained from implanted devices or non-implanted devices. Intra-procedural acquisition may result from stimulation of the heart using a stimulation method or from spontaneous occurring arrhythmias. In both instances, the ECG signals are collected using a multi-lead ECG set up, e.g., with each lead positioned to collect a respective electrocardiogram signal from a different portion of the bodily organ. Signal digitization may be required if the acquired signal has not yet been digitized. At a block 206, signal processing may be applied subsequent to the acquisition, in order to extract the feature set used by the classification algorithm. This signal processing may include: signal segmentation to extract the QRS, signal resampling to map the QRS to a fixed length sample sequence, signal normalization to equalize the total peak-to peak amplitude or the rms amplitude of the ECG signals, morphologic feature extraction to quantify features such as slopes, curvatures, or peak and valley locations from the QRS that might be used as a classifier.

At blocks 202 and 204, a sufficient amount of data is collected such that multi-lead ECG recordings of an arrhythmia (i.e., VT or another target signal) may be obtained. At the block 206, an initial signal analysis may be performed to define signals of interest for analysis, which may include identifying a region of interest of the electrocardiographic signal (i.e., the QRS complex) that will be marked. Image data can also be provided to the block 206, for example, to enhance the classification performance. Such imaging data may be in the form of computed tomography or echocardiography or magnetic resonance imaging or nuclear imaging, as well as scar imaging. Image data may include a series of two-dimensional (2D) or three-dimensional (3D) regions of differing sizes and contours for the bodily organ. These regions may be defined with the use of pace maps, where the size of these regions may be determined by an algorithm analysis to increase accuracy of VT exit site measurement and to increase sensitivity and specificity for ROC curves, and where the size of these regions may be reported as coordinates, areas or volumes.}

At a block 208, the processed patient's ECG data, called “test signal” data in blocks 202 and 204 is imported into an analyzer (e.g., analysis algorithm) after extraction and processing in block 206. Reference signal data from a database 212 is also imported. At block 210, the test data is provided to a region classifier algorithm, described above, which has been determined from the reference data (e.g., from database 212), called “training data” in supervised machine learning terminology, that were obtained during previous pace-mapping and other sampling procedures. This training data may include procedures performed on the test patient, for example. Block 210 uses a classification algorithm to assign likelihood scores to each anatomical region. For example, in a relevance vector machine (RVM) classifier the likelihood score is the posterior probability, computed by the RVM from the measured ECG data, that the signal is associated with anatomical region. For example, the supervised learning method may be applied on shape features of the electrocardiogram signals at predetermined pacing locations. Those shape features of each electrocardiogram signal may be extracted using an energy normalization technique and a signal interpolation technique. The supervised learning method may be implemented through support vector machines, relevance vector machines, neural networks, and/or logistic regression classifiers. The block 210 may make the “test signal” comparison using by comparing electrocardiogram signals based on the QRS complex of the electrocardiogram signals, the P-wave of the electrocardiogram signals, the T-wave of the electrocardiogram signals, any interval or signal of the electrocardiogram during an arrhythmia or during a baseline rhythm, or any combination thereof. In other examples, the block 210 may implement unsupervised learning techniques from un-trained, or unlabeled data from blocks 202 and 204.

After assigning a likelihood score to the ECG signals at block 210, the process 200 (block 214) identifies single or multiple regions (i.e., anatomical areas), and sub-regions that best match the comparison ECG data from block 210. The block 214 may access predetermined anatomical regions stored in a database 215.

At the block 214, the process 200 assigns the ECG data to these regions or sub-regions, e.g., by identifying specific coordinates within these regions and sub-regions. Thus, via the block 214, the process 200 assigns VT exit sites to one of the regions and sub-regions. Via the block 214, the process 200 may determine the anatomical regions, and, for at least one of the anatomical regions, a plurality of sub-regions within the region, for example, using a machine learning technique applied to the mapping data. Accuracy can be further adjusted by providing imaging data (for example computer tomography, magnetic resonance imaging, fluoroscopy, echocardiography or nuclear imaging) that may correct for patient specific parameters (for example, body position, orientation of the heart within the chest, presence or absence of structural heart disease and scar location etc.) That imaging data may be provided to block 214 from an imaging source or database 215 or to any other of the preceding blocks of process 200. In any event, a predetermined set of anatomical regions may be determined from reference electrocardiogram derived signals collected from the plurality of multiple leads from the patient or from a set of patients, where that set may include only different patients or the different patients and the patient under examination. It is noted that the anatomical region may reflect an external region of the bodily organ or an internal region; and, as such, the block 214 may identify VT exit sites at an external or internal region of the organ.

The process 200 constructs and displays the data at a block 218. For example, via the block 218, an image of the bodily organ (e.g., heart), with the anticipated origin of the ventricular arrhythmia or the mapped VT exit site derived from the ECG data, may be constructed and displayed. The image construction and display may occur simultaneously for all VT exit sites that are analyzed. In some examples, the ECG data may be adjusted (at an optional block 216) based on image data and pace-mapping data that are provided to the analysis system, to provide further accuracy. Signal data that has been recorded or generated by pace-mapping in a precisely defined location can be used to better localize the site of origin or the exit site of a particular arrhythmia in a particular patient by the use of neural networks, support vector machines, relevance vector machines, and/or logistic regression classifiers. In some examples, for image integrity, image data may be buffered for display in a more smoothed manner. In some examples, averaging may be embedded in the initial ECG signal collection and analysis. Example images are shown in FIGS. 4A and 4B.

While particular examples are described, it will be appreciated that the techniques may be implemented in various ways to identify and localize physical characteristics. For example, while pace-mapping has been described, any type of suitable mapping may be used by deriving data from historical or contemporaneous electrocardiogram signal data. Furthermore, while identifying of anatomical regions is described, it is noted that such identification includes identifying a particular entity on the bodily organ, attributable to the physical characteristic. That is the anatomical region may be a biological feature of the bodily organ.

With reference to FIG. 5, an exemplary system 300 for implementing the blocks of the method and apparatus includes a general-purpose computing device in the form of a computer 312. The computer 312 may be a ventricular arrhythmia/VT analysis and mapping system. Components of computer 312 may include, but are not limited to, a processing unit 314 and a system memory 316. The computer 312 may operate in a networked environment using logical connections to one or more remote computers, such as remote computers 370-1, 370-2, . . . 370-n, via a first communication network 372, such as local area network (LAN), and/or a second communication network 373, such as wide area network (WAN) 373, via a communication interface 375. The communication interface 375 may include a variety of hardware for wireless and/or wired communications capabilities. Exemplary wireless communication hardware in the communication interface 375 may include cellular telephony circuitry, GPS receiver circuitry, Bluetooth circuitry, Radio Frequency Identification (RFID) or Near Field Communication (NFC) circuitry, and/or Wi-Fi circuitry (i.e., circuitry complying with an IEEE 802.11 standard), as well as hardware supporting any number of other wireless communications protocols. The communication networks 372 and 373 may be over wireless or wired communication links. Example wired communications may include, for example, USB circuitry, Ethernet circuitry, and/or hardware supporting any number of other wired communications protocols. The network 373 may connect the system 312 to any number of network-enabled devices. The remote computers 370-n may represent a network-enabled wireless terminal, a phone, a tablet computer or personal digital assistant (PDA), a smartphone, a laptop computer, a desktop computer, a tablet computer, hospital terminal or kiosk, a portable media player, an e-reader, or other similar devices (not shown). An example smartphone 380 is shown. Of course, any network-enabled device appropriately configured may interact with the system 300. Such devices may be used to display the anatomical regions with identified VT exit sites, for example, via communicating imaging data to a remote device 370-n, 380, etc. having a display for displaying operation of the block 218. Example resulting images are shown in FIG. 4A, which depicts a first set of VT exit sites 250 (only some of which are labeled) mapped to a region of the heart (e.g., region J 120), over an inferior aspect of the left ventricle in a patient with a large inferior wall infarction. The low voltage area was 242 cm². Area J (tags measuring 17.4 cm²) and area I (tags measuring 6.7 cm²) are displayed they identify the region where pace-mapping generated similar signals. Location of the left ventricular apex and mitral valve annulus (MVA) are labeled 252 and 254, respectively. FIG. 4B is an electroanatomical map of sites where pace-mapping was performed, showing identified VT exit sites 256 (only some of which are labeled).

The remote computers 370 may include other computers like computer 312, but in some examples, these remote computers 370 include one or more of (i) an electrocardiogram (ECG) machine, (ii) a medical imaging system, and (iii) a signal records database systems, (iv) a scanner, and/or (v) a signal filtering system

In the illustrated example, the computer 312 is connected to a multi-lead ECG apparatus, labeled machine 370-1. The ECG machine 370-1 may be a stand-alone system, having a multi-lead sensor, such as a 312 lead ECG apparatus described above and a processing machine for performing ECG operation, including transmitting stimulation signals, collecting ECG signals at a user selected scan rate, performing signal analysis on collected ECG signals, such as noise filtering, signal averaging, etc., and storing (and/or buffering) those ECG signals and transmitting the same to the computer 312 for further analysis and pace mapping. In other examples, a multi-lead ECG probe (as described above) may be connected directly to the computer 312, which would then control operation of the multi-lead ECG probe, perform the data processing and storage functions, in place of the remote system 370-1.

Computer 312 typically includes a variety of computer readable media that may be any available media that may be accessed by computer 312 and includes both volatile and nonvolatile media, removable and non-removable media. The system memory 316 includes computer storage media in the form of volatile and/or nonvolatile memory such as read only memory (ROM) and random access memory (RAM). The ROM may include a basic input/output system (BIOS). RAM typically contains data and/or program modules that include operating system 320, application programs 322, other program modules 324, and program data 326. The memory 316 may store instructions that when executed by the processor 314 perform ventricular arrhythmia/VT analysis and pace-mapping and other techniques in accordance with the examples described here, for example, stored as the programs 322 and 324, and implementing the process 200. The computer 312 may also include other removable/non-removable, volatile/nonvolatile computer storage media such as a hard disk drive, a magnetic disk drive that reads from or writes to a magnetic disk, and an optical disk drive that reads from or writes to an optical disk.

A user may enter commands and information into the computer 312 through input devices such as a keyboard 330 and pointing device 332, commonly referred to as a mouse, trackball or touch pad. Other input devices (not illustrated) may include a microphone, joystick, game pad, satellite dish, scanner, or the like. These and other input devices are often connected to the processing unit 314 through a user input interface 335 that is coupled to a system bus, but may be connected by other interface and bus structures, such as a parallel port, game port or a universal serial bus (USB). A monitor 340 or other type of display device may also be connected to the processor 314 via an interface, such as a video interface 342. In addition to the monitor, computers may also include other peripheral output devices such as speakers 350 and printer 352, which may be connected through an output peripheral interface 355.

Generally, the techniques herein may be coded any computing language for execution on computer 312. ECG data may be obtained from the remote computers 370-1, 370-2, . . . 370-n and stored loaded on to any of the computer storage devices of computer 312. Once the ECG data is obtained, a user may input or select the condition parameters through an input mechanism as described. Although, in other examples, the condition parameters may be pre-selected or automatically determined, for example, based on a particular type of analysis that is to be performed. The output of the executable program may be displayed on a display (e.g., a monitor 340), sent to a printer 352, stored for later use by the computer 312, or offloaded to another system, such as one of the remote computers 370. The output may be in the form of an image (such as FIGS. 4a and 4b), a graph, a table or any combination thereof, by way of example. Operations of the system may be recorded in a log database for future reference as shown. This log database may be accessed at subsequent times. In any event, the VT exit site analysis and pace mapping described herein is implemented on the computer 312, in the illustrated example.

It will be appreciated that the above descriptions are provided by way of example and that numerous modifications may be made within context of the present techniques. Therefore, while the techniques herein provide for determining a correspondence between the electrocardiogram derived signals and the anatomical area containing the physical characteristic from a training set of pacemap data, a supervised learning method of various kinds may be used to implement the techniques. The supervised learning method may be implemented through support vector machines, relevance vector machines, neural networks, and/or logistic regression classifiers, for example. The supervised learning method may be applied on shape features of the electrocardiogram signals and predetermined pacing locations, where those shape features of each electrocardiogram signal are extracted using an energy normalization technique and a signal interpolation technique. As an example, energy normalization can be accomplished by dividing each signal by its RMS average amplitude; and signal interpolation can be accomplished by applying splines, cardinal series, or the wavelet transform. And in other examples, the supervised learning method may be replaced with an unsupervised learning method.

More generally, the various blocks, operations, and techniques described above may be implemented in hardware, firmware, software, or any combination of hardware, firmware, and/or software. When implemented in hardware, some or all of the blocks, operations, techniques, etc. may be implemented in, for example, a custom integrated circuit (IC), an application specific integrated circuit (ASIC), a field programmable logic array (FPGA), a programmable logic array (PLA), etc.

When implemented in software, the software may be stored in any computer readable memory such as on a magnetic disk, an optical disk, or other storage medium, in a RAM or ROM or flash memory of a computer, processor, hard disk drive, optical disk drive, tape drive, etc. Likewise, the software may be delivered to a user or a system via any known or desired delivery method including, for example, on a computer readable disk or other transportable computer storage mechanism or via communication media. Communication media typically embodies computer readable instructions, data structures, program modules or other data in a modulated data signal such as a carrier wave or other transport mechanism. The term “modulated data signal” means a signal that has one or more of its characteristics set or changed in such a manner as to encode information in the signal. By way of example, and not limitation, communication media includes wired media such as a wired network or direct-wired connection, and wireless media such as acoustic, radio frequency, infrared and other wireless media. Thus, the software may be delivered to a user or a system via a communication channel such as a telephone line, a DSL line, a cable television line, a wireless communication channel, the Internet, etc. (which are viewed as being the same as or interchangeable with providing such software via a transportable storage medium).

Moreover, while the present invention has been described with reference to specific examples, which are intended to be illustrative only and not to be limiting of the invention, it will be apparent to those of ordinary skill in the art that changes, additions and/or deletions may be made to the disclosed embodiments without departing from the spirit and scope of the invention.

Thus, although certain apparatus constructed in accordance with the teachings of the invention have been described herein, the scope of coverage of this patent is not limited thereto. On the contrary, this patent covers all embodiments of the teachings of the invention fairly falling within the scope of the appended claims either literally or under the doctrine of equivalents.

What is claimed is:

1. A method for identifying a physical characteristic and localizing characteristics of sites within a bodily organ, the method comprising:
collecting electrocardiogram derived signals from a plurality of multiple electrical leads, each lead positioned to collect a respective electrocardiogram signal from different sites of the bodily organ;
comparing the collected electrocardiogram derived signals to reference signal data to identify which anatomical region among a predetermined set of anatomical regions corresponding to the bodily organ contains the physical characteristic, wherein the reference signal data is determined from previously-labeled electrocardiogram derived signals for one or more different anatomic areas of the bodily organ; and
using mapping data derived from the subsequently collected electrocardiogram derived signals to determine a sub-region within the anatomical region that contains the physical characteristic.

2. The method of claim 1, wherein the mapping data is pace-mapping data or spontaneously occurring arrhythmia data.

3. The method of claim 1, further comprising:
determining the predetermined set of anatomical regions; and
determining, for at least one of the anatomical regions, a plurality of sub-regions within the region using a machine learning technique applied to the mapping data.

4. The method of claim 3, further comprising determining a correspondence between the electrocardiogram derived signals and the anatomical region containing the physical characteristic from a training set of pace-mapping data or data from spontaneously occurring arrhythmias.

5. The method of claim 3, further comprising applying a supervised learning method on shape features of the electrocardiogram signals and at predetermined pacing locations.

6. The method of claim 5, wherein the shape features of each electrocardiogram signal are extracted using an energy normalization technique and a signal interpolation technique.

7. The method of claim 5, wherein the supervised learning method is implemented through support vector machines, relevance vector machines, neural networks, and/or logistic regression classifiers.

8. The method of claim 1, wherein the bodily organ is the heart and the multiple leads are leads of a multi-lead electrocardiogram assembly.

9. The method of claim 1, further comprising displaying an image of the bodily organ indicating physical characteristic inducing sites assigned to the sub-regions.

10. The method of claim 9, wherein the physical characteristic inducing sites are ventricular tachycardia or other arrhythmia sites.

11. The method of claim 1, wherein the predetermined set of anatomical regions includes regions of differing sizes and contours for the bodily organ.

12. The method of claim 11, further comprising determining the predetermined set of anatomical regions from reference electrocardiogram derived signals collected from the plurality of multiple leads.

13. The method of claim 1, further comprising determining the predetermined set of anatomical regions from reference electrocardiogram signals collected from a plurality of different patients.

14. The method of claim 1, wherein the electrocardiogram signals are compared based on the QRS complex of the electrocardiogram signal, the P-wave of the electrocardiogram signal, the T-wave of the electrocardiogram signal, any interval or signal of the electrocardiogram during an arrhythmia or during a baseline rhythm, or any combination thereof.

15. An apparatus comprising:
a computer processor; and
a memory storing computer-readable instructions that, when executed by the computer processor, cause the computer processor to,
collect electrocardiogram derived signals from a plurality of multiple electrical leads, each lead positioned to collect a respective electrocardiogram signal from different stimulated sites of the bodily organ,
compare the electrocardiogram derived signals to reference signal data to identify, among a set of predetermined anatomical regions of the bodily organ, anatomical regions that contain a physical characteristic of the bodily organ, wherein the reference signal data is determined from previously-labeled electrocardiogram derived signals for one or more different anatomic areas of the bodily organ, and
use mapping data derived from the electrocardiogram derived signals to determine a sub-region within the anatomical region that contains the physical characteristic.

16. The apparatus of claim 15, wherein the mapping data is pace-mapping data.

17. The apparatus of claim 15, wherein the memory stores further computer-readable instructions that, when executed by the computer processor, cause the computer processor to:
determine the predetermined set of anatomical regions; and
determine, for at least one of the anatomical regions, a plurality of sub-regions within the region using a machine learning technique applied to the mapping data.

18. The apparatus of claim 17, wherein the memory stores further computer-readable instructions that, when executed by the computer processor, cause the computer processor to determine a correspondence between the electrocardiogram derived signals and the anatomical region containing the physical characteristic from a training set of pacemap data.

19. The apparatus of claim 17, wherein the memory stores further computer-readable instructions that, when executed by the computer processor, cause the computer processor to apply a supervised learning method on shape features of the electrocardiogram signals and at predetermined pacing locations.

20. The apparatus of claim 19, wherein the shape features of each electrocardiogram signal are extracted using an energy normalization technique and a signal interpolation technique.

21. The apparatus of claim 19, wherein the supervised learning method is implemented through support vector machines, relevance vector machines, neural networks, and/or logistic regression classifiers.

22. The apparatus of claim 15, wherein the bodily organ is the heart and the multiple leads are leads of a multi-lead electrocardiogram assembly.

23. The apparatus of claim 15, wherein the memory stores further computer-readable instructions that, when executed by the computer processor, cause the computer processor to display an image of the bodily organ indicating physical characteristic inducing sites assigned to the sub-regions.

24. The apparatus of claim 23, wherein the physical characteristic inducing sites are ventricular tachycardia or other arrhythmia sites.

25. The apparatus of claim 15, wherein the predetermined set of anatomical regions includes regions of differing sizes and contours for the bodily organ.

26. The apparatus of claim 25, wherein the memory stores further computer-readable instructions that, when executed by the computer processor, cause the computer processor to determine the predetermined set of anatomical regions from reference electrocardiogram derived signals collected from the plurality of multiple leads.

27. The apparatus of claim 15, wherein the memory stores further computer-readable instructions that, when executed by the computer processor, cause the computer processor to determine the predetermined set of anatomical regions from reference electrocardiogram signals collected from a plurality of different patients.

28. The apparatus of claim 15, wherein the electrocardiogram signals are compared based on the QRS complex of the electrocardiogram signal, the P-wave of the electrocardiogram signal, the T-wave of the electrocardiogram signal, any signal or interval of the electrocardiogram taken during an arrhythmia or during baseline rhythm, or any combination thereof.

29. The apparatus of claim 15, wherein the memory stores further computer-readable instructions that, when executed by the computer processor, cause the computer processor to build a database of labeled electrocardiogram derived signals, wherein the labeled electrocardiogram derived signals form reference signal data for one or more different anatomic areas of the bodily organ.
